(19) United States
(12) Patent Application Publication
(10) Pub. No.: US 2022/0355290 A1
(43) Pub. Date: Nov. 10, 2022

(54) SAMPLE COLLECTION DEVICE AND METHODS OF USING SAME

(71) Applicant: Exosome Diagnostics, Inc., Waltham, MA (US)

(72) Inventors: Georg STOLL, Munich (DE); Frank NUSS, Bargen (DE); Daniel ENDERLE, Martinsried (DE); Mikkel NOERHOLM, Martinsried (DE)

(21) Appl. No.: 17/621,084

(22) PCT Filed: Jun. 24, 2020
(86) PCT No.: PCT/US2020/039286

§ 371 Date: Dec. 20, 2021

Related U.S. Application Data
(60) Provisional application No. 62/867,744, filed on Jun. 27, 2019.

(51) Int. Cl. B01L 3/00; A61B 10/00

(52) U.S. Cl. CPC B01L 3/502; A61B 10/007; B01L 2200/0684; B01L 2300/047; B01L 2300/048; B01L 2300/123; B01L 2300/08

(57) ABSTRACT

Described herein are various inventions and embodiments thereof, directed to systems, devices, and methods for separating a sample. Embodiments of sample collection devices disclosed herein may maintain separation of different volumes of sample during collecting, handling, and accessing of the sample. A sample collection device, in some embodiments, may comprise an enclosure defining a first chamber, a second chamber, and a partition therebetween configured to separate the first chamber and the second chamber. An assembly coupled to the partition may be configured to transition between an open configuration and a closed configuration based on fluid and gas flow through the assembly.

### RELATED APPLICATIONS

This application claims priority to, and the benefit of, U.S. Provisional Application No. 62/867,744, filed Jun. 27, 2019, the contents of which are incorporated herein by reference in their entirety.

### TECHNICAL FIELD

Devices, systems, and methods herein relate to sample collection containers that may be used in diagnostic applications, including but not limited to urine collection and analysis.

### BACKGROUND

Analysis of a biofluid sample from a subject may be used as a diagnostic tool for disease and to monitor subject health. In some cases, a first portion of a sample, (e.g., an initial first catch of sample volume) may contain the largest quantity of components of interest in the sample and may be particularly useful for analysis. For example, the first 20 ml to 40 ml of urine from a patient may contain the greatest concentration of hormones and exosomal RNA prostate cancer markers to be analyzed in the urine sample.

Some conventional sample collection devices retain only the first catch of sample volume without saving any extra volume (e.g., second catch) may require additional sample collection steps and/or collection devices in order to perform additional tests. Other conventional sample collection devices are configured to collect and separate a first catch from any remaining volume (e.g., second catch) of the sample, but may be prone to mixing and spilling during processing and handling.

Therefore, additional devices, systems, and methods of sample collection may be desirable.

### SUMMARY

Described herein are inventions and embodiments of sample collection devices, systems, and methods, including the features, structure, functionality and steps thereof, for collection and separation of portions of a biofluid sample such as urine. Embodiments of the sample collection devices disclosed herein may allow collection of a first catch and second catch volume of sample in respective first and second chambers of the device. The sample collection device may be handled and processed without mixing fluid volumes or spilling. Either chamber of the device may be accessed for clinical testing while maintaining the separation between the first catch and the second catch.

Generally, the systems and methods described herein may include a sample collection device configured to receive a sample. The device may include a flexible member configured to function as a one-way valve that may allow fluid to flow into a first chamber from a second chamber through at least one port. The flexible member functions to resist fluid flow back into the second chamber. The port may have an opening configured to allow unrestricted fluid flow. The flexible member may be configured to have the flexibility to allow fluid flow through the port with sufficient rigidity to prevent fluid from passing back into the second chamber. The device may further include a vent configured to allow one or more gases (e.g., air) to flow between chambers in response to sample flowing from the second chamber into the first chamber. The vent may further be configured to resist flow of the first catch from the first chamber to the second chamber. For example, once a level of the fluid in the first chamber reaches the vent, gas will be prohibited from flowing through the vent from the first chamber to the second chamber, thereby preventing fluid flow between chambers. The shape of the partition may aid retention of the first volume of sample within the first chamber.

In some embodiments, a sample collection device is provided, comprising an enclosure defining a first chamber, a second chamber, and a partition therebetween configured to separate the first chamber and the second chamber. At least one port may be coupled to the partition. A vent may be configured to allow one or more gases to escape from the first chamber. A flexible member may be coupled to the at least one port and configured to transition between an open configuration and a closed configuration based on fluid flow through the port and gas flow through the vent.

In some embodiments, a sample collection device is provided, comprising an enclosure defining a first chamber, a second chamber, and a partition therebetween configured to separate the first chamber and the second chamber. An assembly may comprise at least one port, a sealing member, a vent, and a flexible member and may be configured to couple to a shoulder of the partition, the flexible member configured to transition between an open configuration and a closed configuration based on fluid and gas flow through the assembly.

In some embodiments, a sample collection device is provided, comprising an enclosure defining a first chamber, a second chamber, and a partition therebetween configured to separate the first chamber and the second chamber. At least one port may be coupled to the partition. A vent may be configured to allow one or more gases to escape from the first chamber. A lid may comprise a protrusion configured to seal one or more of the vent and the at least one port from the second chamber when the lid is coupled to the enclosure.

In some embodiments, the open configuration may be configured to permit fluid flow of a predetermined volume of sample from the second chamber to the first chamber. The closed configuration may be configured to resist fluid and gas flow from the first chamber to the second chamber.

In some embodiments, in the open configuration, one or more gases may flow out of the first chamber toward the second chamber via the vent during the fluid flow from the second chamber into the first chamber. In some embodiments, the flexible member in the open configuration bends away from the at least one port and the flexible member in the closed configuration forms a seal over the at least one port. In some embodiments, the flexible member may be configured to form a seal over the at least one port when the first chamber holds a predetermined volume of sample.

In some embodiments, the partition comprises a flat portion, and the flexible member may be configured to form a seal in contact with the flat portion when the first chamber holds a predetermined volume of sample. In some embodiments, the partition may define at least one recess around a circumference of the at least one port. In some embodiments, the partition comprises a central recess portion.

In some embodiments, the flexible member has a thickness of between about 0.1 mm and about 1 mm. In some embodiments, the flexible member has a diameter of between about 20 mm and about 30 mm. In some embodiments, the flexible member may be composed of silicone.

In some embodiments, the vent may be configured to permit gas flow from the first chamber to the second chamber during fluid flow into the first chamber. In some embodiments, the vent may define a lumen. In some embodiments, the vent has a diameter of at least about 5 mm. In some embodiments, the enclosure defines a longitudinal axis and the vent is substantially parallel to the longitudinal axis. In some embodiments, the vent extends into at least one of the first chamber and the second chamber. In some embodiments, the vent extends into the first chamber by up to about 15 mm. In some embodiments, the vent extends into the second chamber, and a sidewall of the vent defines an aperture. In some embodiments, the vent comprises a protrusion and the flexible member is coupled to the vent between the partition and the protrusion.

In some embodiments, the flexible member comprises a material inert to biological matter. In some embodiments, the partition extends toward the first chamber. In some embodiments, the partition may be concave. In some embodiments, the first chamber may be configured to hold a sample volume of between about 5 ml and about 50 ml. In some embodiments, the second chamber may be configured to hold a sample volume of between about 10 ml and about 150 ml.

In some embodiments, a first lid may be configured to removably couple to a first end of the enclosure. In some embodiments, a second lid may be configured to removably couple to a second end of the enclosure. In some embodiments, the second lid may be coupled to the enclosure is configured to form a seal between the first chamber and the second chamber.

In some embodiments, the second lid may comprise a protrusion configured to seal one or more of the vents and the at least one port from the second chamber when the second lid is coupled to the enclosure. In some of these embodiments, the protrusion defines a recess configured to accommodate the vent. In some embodiments, the port defines a recess configured to engage a sealing member.

In some embodiments, a sample collection device is provided, comprising an enclosure defining a first chamber, a second chamber, and a partition therebetween configured to separate the first chamber and the second chamber. The enclosure defines a longitudinal axis. The partition may be concave and extend toward the first chamber. At least one port may be coupled to the partition and a sealing member therebetween. A flexible member may be coupled to the at least one port. A vent may be coupled to the flexible member. The vent may define a lumen and extend substantially parallel to the longitudinal axis. The vent may comprise a first protrusion and the flexible member may be coupled between the partition and the protrusion. A first lid may be coupled to a first end of the enclosure. A second lid comprises a second protrusion configured to seal one or more of the vent and the at least one port from the second chamber when the second lid is coupled to a second end of the enclosure. The second protrusion defines a recess configured to accommodate the vent.

Also described here are embodiments corresponding to sample collection method. In general, these methods may include the steps of receiving a first volume of sample into a sample collection device. The sample collection device may comprise an enclosure defining a first chamber and a second chamber. The enclosure may comprise a flexible member and a partition configured to separate the first chamber and the second chamber. The first volume may be received in the second chamber. The first volume may flow from the second chamber to the first chamber such that the flexible member bends. A second volume of the sample may be received in the second chamber. Fluid and gas flow between the first chamber and the second chamber may be resisted such that the first volume in the first chamber is substantially separated from the second volume in the second chamber.

In some embodiments, the flexible member may transition between an open configuration and a closed configuration based on fluid and gas flow. The open configuration permits fluid and gas flow from the second chamber to the first chamber. The closed configuration resists fluid and gas flow from the first chamber to the second chamber.

In some embodiments, the sample collection device may be handled while maintaining the separation between the first volume and the second volume. In some embodiments, the first chamber may be sealed from the second chamber by coupling a lid to the enclosure. In some embodiments, gas may be vented from the first chamber to the second chamber.

In some embodiments, at least a portion of the sample may be accessed from one of the first chamber and the second chamber while maintaining separation between the first volume and the second volume. In some of these embodiments, accessing at least the portion of the first volume from the first chamber comprises tilting the sample collection device. In some embodiments, the sample is urine.

These and other embodiments, advantages and objects of the present disclosure will be even better understood with reference to the detailed description.

### BRIEF DESCRIPTION OF THE DRAWINGS

FIGS. 1A-1D are illustrative views of a sample collection device according to some embodiments of the disclosure. FIGS. 1A and 1B are perspective views of the device, FIG. 1C is a side view of the device without top and bottom lids, and FIG. 1D is a perspective view of a lower portion of the device without a lid.

FIGS. 2A and 2B are illustrative side cross-sectional views of a sample collection device according to some embodiments of the disclosure.

FIGS. 3A-3C are illustrative side cross-sectional views of a sample collection device receiving a sample according to some embodiments of the disclosure.

FIGS. 4A-4C are illustrative views of an assembly of a sample collection device according to some embodiments of the disclosure. FIG. 4A is a perspective view of the assembly, FIG. 4B is a side view of the assembly, and FIG. 4C is a plan view of the assembly.

FIGS. 5A-5C are illustrative perspective views of various embodiments of an assembly of the disclosure.

FIGS. 6A and 6B are illustrative side cross-sectional views of a sample collection device according to some embodiments of the disclosure. FIG. 6C is a plan view of the sample collection device, FIG. 6D is a perspective view of the sample collection device, and FIG. 6E is a bottom view of the sample collection device.

FIG. 7A is an illustrative perspective view of a lid according to some embodiments of the disclosure. FIG. 7B is a side view of the lid, and FIG. 7C is a plan view of the lid.

FIG. 8 is an illustrative perspective view of a lid according to some embodiments of the disclosure.

FIG. 9 is an illustrative flowchart of a sample collection method according to some embodiments of the disclosure.

### DETAILED DESCRIPTION

Described herein are embodiments of systems, devices, and methods for separating a sample using a sample collection device. In some embodiments, the sample collection device is configured to collect and separate a first volume of sample from a second volume of sample during collection and handling of the sample. The first and second volumes of the sample may be received continuously by the device. The device may maintain sample separation while being accessed so to allow a user (e.g., lab technician) to access at least one of the sample volumes by for example, aspirating the sample using a syringe or pouring the sample into a separate container. Furthermore, the device may be formed of materials that do not interfere with analysis of the sample. For example, the device does not leak substances into the sample in a manner that alters and/or degrades the sample.

In some embodiments, a first volume of sample may be initially received into a second chamber of a sample collection device. For example, a patient may urinate into an open end of the device at any suitable angle and flow rate without loss of sample. An assembly within the device may allow the first volume to flow from the second chamber into the first chamber. For example, the first chamber may hold about 25 ml of fluid as a first volume of sample. The patient may continue to urinate to fill the second chamber with a second volume of sample. When a predetermined volume of the sample is collected in the first chamber, the assembly may resist fluid and gas flow between the first chamber and the second chamber such that the volume in the first chamber is substantially separated from the second volume in the second chamber. A lid may be placed over the open end to seal the device. The device may be handled, transported, and otherwise manipulated while maintaining sample separation between the chambers without spilling or leaking. For example, the device may be shaken, rotated, and placed on a surface for a predetermined time period. The device may be configured to allow one or more labels to be provided on the device for identification of one or more of the sample, patient, and tests. For example, a label may be affixed over the sidewalls and lid of the device to form a tamper proof seal. When a lid on either end of the device is removed, the collected sample volume may be freely accessible for sample analysis (e.g., dipstick, microscopy, microbiological analysis). For example, one or more portions of a sample may be poured into another container (e.g., centrifuge tube, syringe). For urine samples collected by the device, exosomes and other urine constituents do not undergo adsorption or degradation. Furthermore, EPI, qPCR, and/or analyte signals derived from the sample are not altered by the materials forming the sample collection device.

### I. Devices

Described herein are devices that may be used in some embodiments of the various systems described. A sample collection device as described herein may comprise an enclosure defining a first chamber, a second chamber, and a partition therebetween configured to separate the first chamber and the second chamber. The enclosure may define a longitudinal axis. The partition may be concave and extend toward the first chamber. At least one port may be coupled to the partition with a sealing member disposed therebetween. A flexible member may be coupled to at least one port. A vent may be coupled to the flexible member. The vent may define a lumen and may extend substantially parallel to the longitudinal axis. The vent may comprise a first protrusion and the flexible member may be coupled between the partition and the protrusion. A first lid may be coupled to a first end of the enclosure. A second lid may comprise a second protrusion configured to seal one or more of the vent and at least one port from the second chamber when the second lid is coupled to a second end of the enclosure. The second protrusion may define a recess configured to accommodate the vent.

### Enclosure

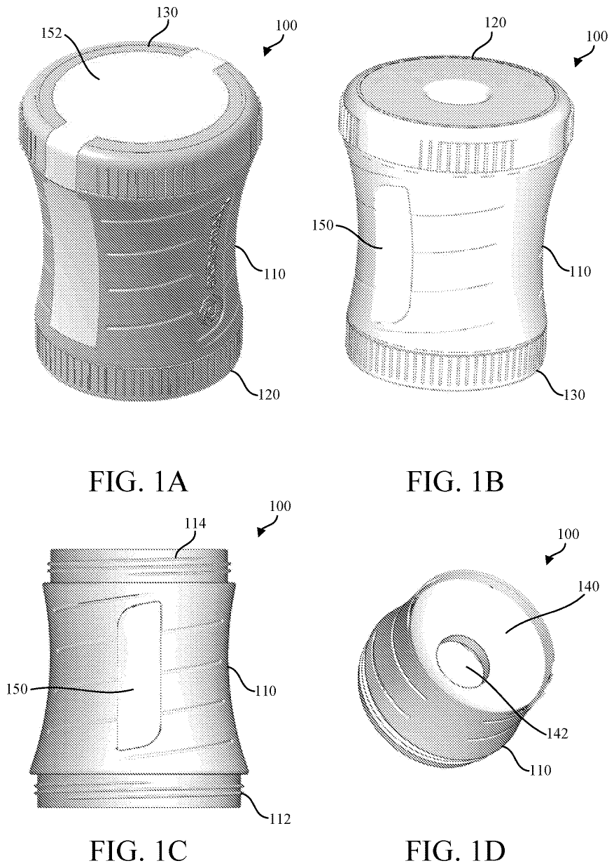

As shown in FIGS. 1A and 1B, a sample collection device (100) may comprise an enclosure (110), a first lid (120) (e.g., bottom lid), and a second lid (130) (e.g., top lid). FIG. 1A illustrates the device (110) with the first lid (120) facing downward and FIG. 1B illustrates the device (110) with the first lid (120) facing upward. In some embodiments, one or more portions of the enclosure (110), first lid (120), and second lid (130) may comprise a label (150, 152). For example, a first label (150) may correspond to a side portion of the enclosure (110), and a second label (152) may extend from a first side portion of the enclosure (110) and across a surface of the second lid (130) and over to a second side portion of the enclosure (110) opposite the first side portion. Additionally or alternatively, an identifier (e.g., barcode sticker) including, for example, a sticker having an adhesive backing, may be placed over the label (150, 152). In some embodiments, the label may include patient data (e.g., sample identification, patient identification, date) and test data.

In some embodiments, one or more portions of the first lid (120) and the second lid (130) may be transparent so as to allow visual inspection of any contents within respective first and second chambers of the device (100).

FIG. 1C illustrates an embodiment of the sample collection device (100) without the first lid (120) and the second lid (130). Each of a first end (112) and a second end (114) of the enclosure (110) may be threaded such that the first lid (120) and the second lid (130) may be removably coupled (e.g., screwed) to the enclosure (110). In some embodiments, the enclosure (110) may have a shape and/or texture configured to aid handling by a user. For example, the surface of one or more of the enclosure (110), first lid (120), and second lid (130) may have a concave shape and/or have a textured surface to aid traction and grip. The shape of the device (100) may also serve as a visual and/or tactile indicator for the user of the designated top and bottom of the device.

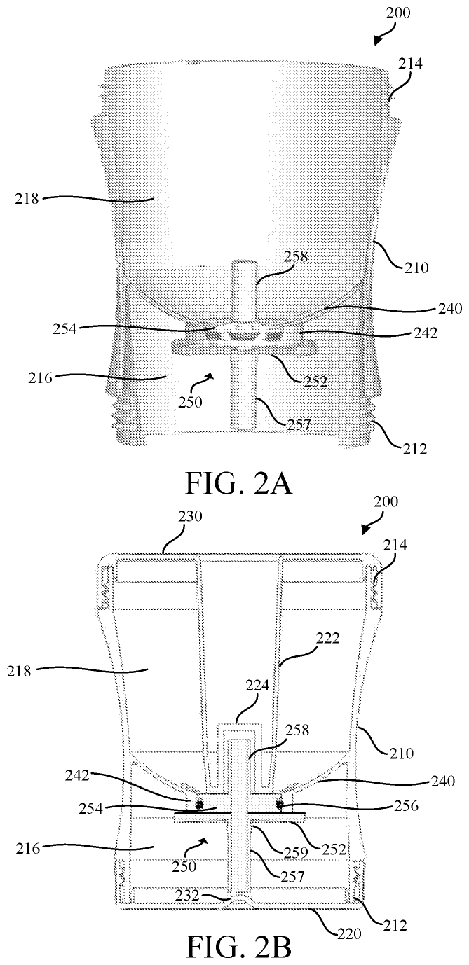

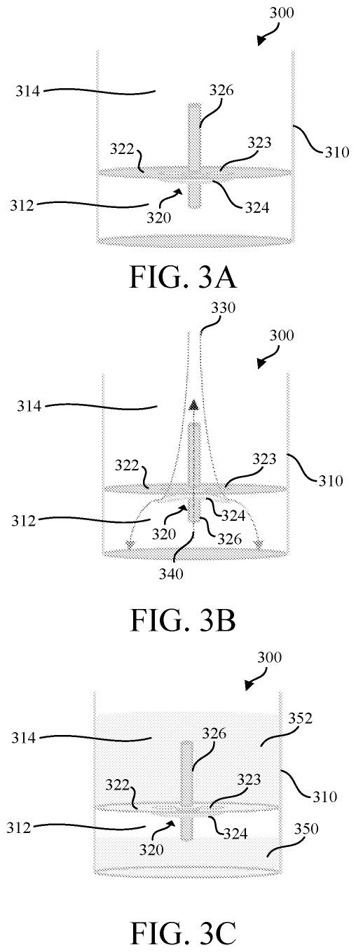

FIG. 1D illustrates a bottom perspective view of the enclosure (110) without the first lid (130). The enclosure (100) may comprise a partition (140) that separates a first chamber and a second chamber of the enclosure (100), as shown for example in FIGS. 2A-2B and 3A-3C. The partition may define an opening (142) configured to couple to an assembly, as described in more detail herein. In some embodiments, an assembly may be removably coupled to the partition (140) so as to allow configuration of a sample collection device for different biofluids and/or sample volumes. In FIG. 1D, the opening (142) is disposed in a center of the partition (140) but may be off-center. In some embodiments, the partition (140) may define a plurality of openings each configured to removably couple to an assembly. For example, a partition may define a set of three openings disposed equidistant from each other and a center of the partition. In some embodiments, the assembly may be formed integral with the partition such as shown in FIGS. 3A-3C.

### Partition

FIG. 2A is a side cross-sectional view of a sample collection device (200) comprising an enclosure (210) and an assembly (250) coupled thereto. As shown in FIG. 2B, a first lid (220) may be removably coupled to the first end (212) and a second lid (230) may be removably coupled to the second end (214). Each of a first end (212) and a second end (214) of the enclosure (210) may be threaded. The enclosure (210) may define a first chamber (216) and a second chamber (218). The enclosure (210) may include a partition (240) disposed therebetween and configured to separate the first chamber (216) and the second chamber (218). The partition (240) may be shaped such that fluid in the second chamber (218) may preferentially flow along the partition (240) toward the assembly (250). For example, the partition (240) may generally extend toward the first chamber (216). The partition (240) may be concave. In FIGS. 2A and 2B, the partition (240) may be shaped so as to form a radial opening between the sidewalls of the enclosure (210) and the partition (240). Alternatively, the partition may be generally planar.

In some embodiments, the partition (240) may include a portion (242) (e.g., shoulder, protrusion) forming a perimeter of the radial opening and configured to engage the assembly (250). For example, a port (254) of the assembly (250) may be held by the portion (242) of the partition (240). A sealing member (256) may be disposed between the portion (242) and the port (254). The sealing member (256) may be, for example, a gasket or O-ring. The interface between the portion (242) and the port (254) may be coupled to form a fluid-tight seal. In some embodiments, the assembly (250) may be snap-fit to the partition (240).

In some embodiments, the first chamber (216) is configured to hold a first sample volume (e.g., first catch volume of urine) of between about 5 ml and about 50 ml, between about 15 ml and about 30 ml, and between about 20 ml and about 30 ml, including all values and sub-ranges in-between. In some embodiments, the second chamber (218) is configured to hold a second sample volume (e.g., second catch volume of urine) of between about 10 ml and about 150 ml, between about 20 ml and about 120 ml, between about 20 ml and about 75 ml, and between about 20 ml and about 75 ml, including all values and sub-ranges in-between. In some embodiments, the second chamber (218) may have larger volume than the first chamber (216).

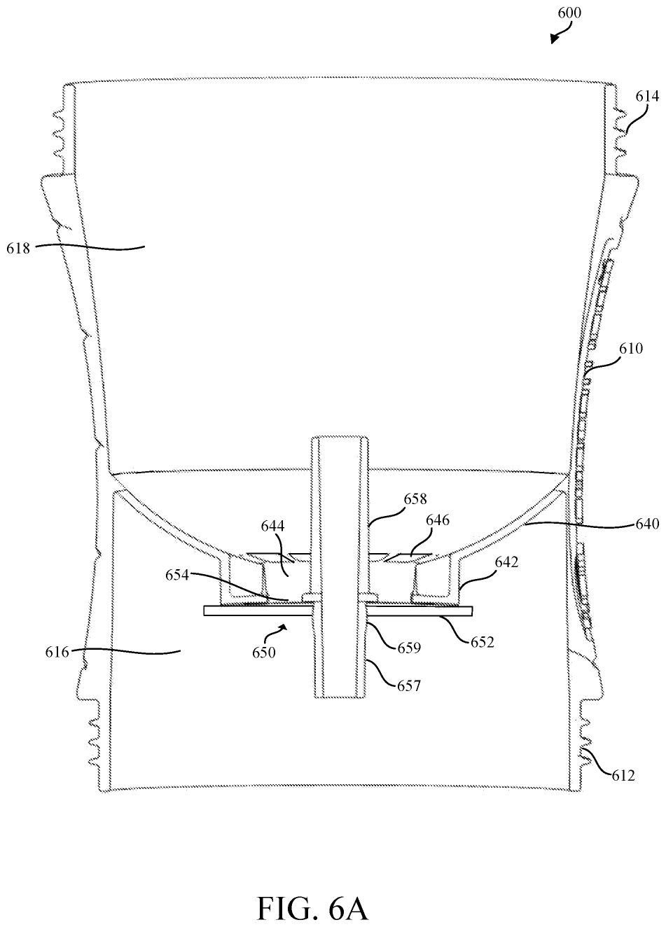

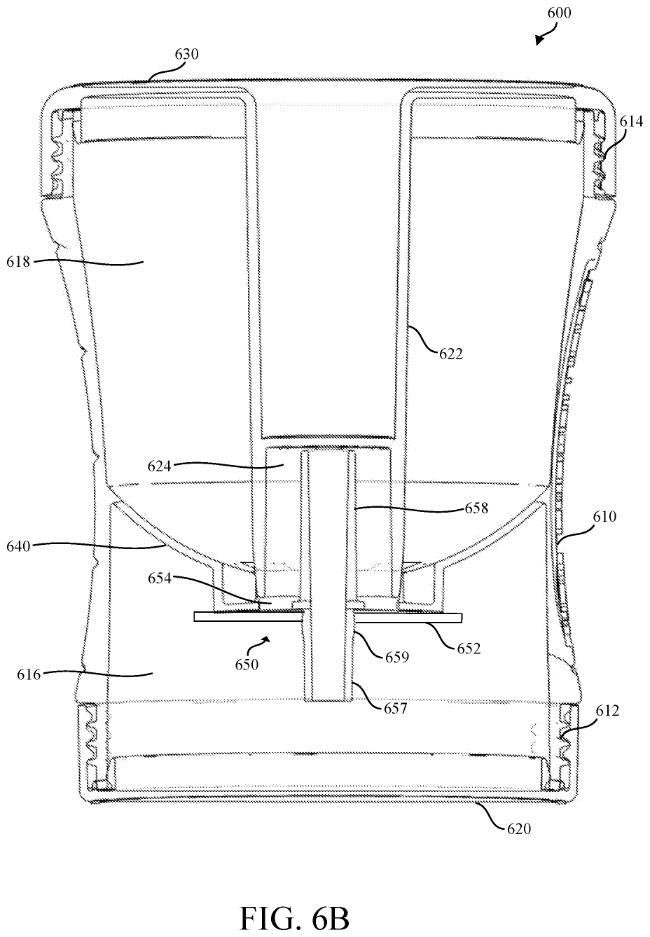

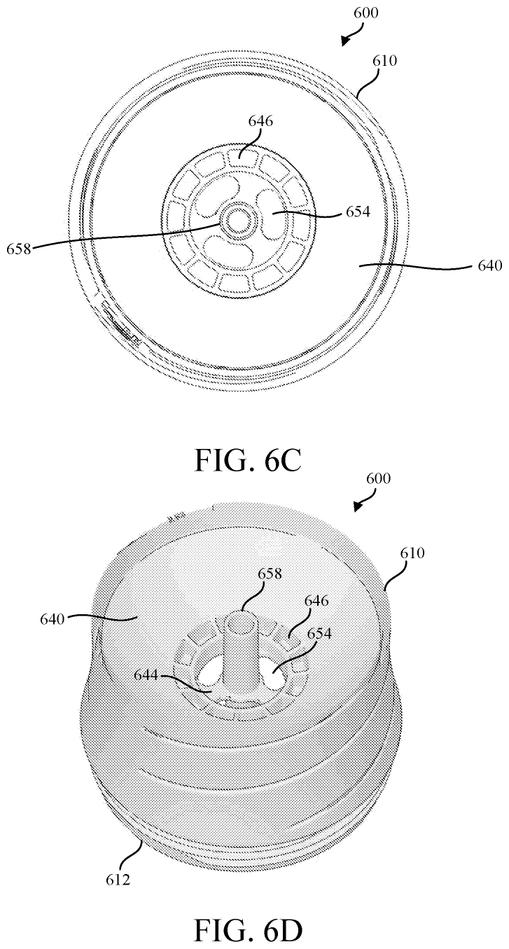

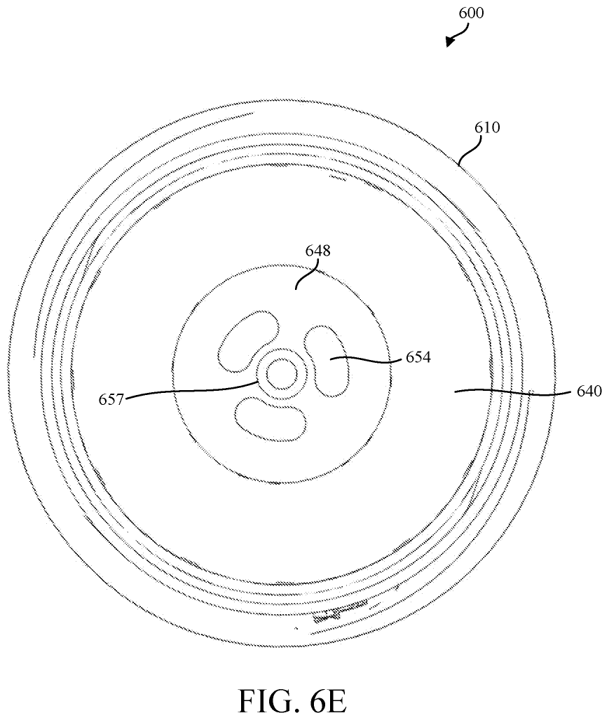

FIG. 6A is a side cross-sectional view of a sample collection device (600) comprising an enclosure (610) and an assembly (650) coupled thereto. As shown in FIG. 6B, a first lid (620) may be removably coupled to the first end (612) and a second lid (630) may be removably coupled to the second end (614). Each of a first end (612) and a second end (614) of the enclosure (610) may be threaded. The enclosure (610) may define a first chamber (616) and a second chamber (618). The enclosure (610) may include a partition (640) disposed therebetween and configured to separate the first chamber (616) and the second chamber (618). The partition (640) may be shaped such that fluid in the second chamber (618) may preferentially flow along the partition (640) toward the assembly (650). For example, the partition (640) may generally extend toward the first chamber (616). The partition (640) may be concave.

As shown in FIG. 6D, the partition (640) may comprise a central recess portion (644) that extends more toward the first chamber (616) than an outer portion (e.g., concave sloped portion) of the partition (640). The central portion (644) may be recessed relative to the other portions of the partition (640). For example, a transition from the outer portion of the partition (640) and the central portion (644) may comprise a steep slope (e.g., shoulder) such that liquid that flows into the central portion (644) is unable to flow out of the central portion (644) absent an external force or a predetermined volume of liquid filling the partition (640). The central portion (644) may be generally circular.

As shown in FIGS. 6C and 6D, a partition (640) may comprise at least one recess (646) configured to increase a volume of the second chamber (618) and hold a predetermined volume of fluid. In some embodiments, the partition (640) may define at least one recess (646) around a circumference of at least one port (654). For example, a plurality of recesses (646) may be disposed proximate to a perimeter of the central portion (654) of the partition (640) so as to form a ring of recesses (646). The partition (640) may comprise a plurality of recesses (646) in order to reduce the uptake of a first catch dead volume. Although the recesses (646) are shown as disposed substantially around the entire circumference of the central portion (644), the recesses (646) may be disposed around less than the entire circumference of the central portion (644).

In some embodiments, enclosure (610), partition (640), and assembly (650) may be formed together in a single piece. For example, the device (600) may be injection molded to reduce the number of manufacturing steps. A flexible member (652) and/or the lids (620, 630) may be assembled after injection-molding the device (600). The sample collection device (600) shown in FIGS. 6C, 6D, and 6E illustrate a set of three ports (654), however, any suitable number of ports, sizes and shapes of ports may be used.

In some embodiments, the first chamber (616) is configured to hold a first sample volume (e.g., first catch volume of urine) of between about 5 ml and about 50 ml, between about 15 ml and about 30 ml, and between about 20 ml and about 30 ml, including all values and sub-ranges in-between. In some embodiments, the second chamber (618) is configured to hold a second sample volume (e.g., second catch volume of urine) of between about 10 ml and about 150 ml, between about 20 ml and about 120 ml, between about 20 ml and about 75 ml, and between about 20 ml and about 75 ml, including all values and sub-ranges in-between. In some embodiments, the second chamber (618) may have larger volume than the first chamber (616).

### Assembly

The assembly (250) coupled to the partition (240) may comprise at least one port (254), a sealing member (256), a vent (257, 258), and a flexible member (252). The port (254) may be coupled to the partition (240). The vent (257, 258) may be configured to allow one or more gases to escape from the first chamber. The flexible member (252) may be coupled to the port (254). The assembly (250) may be removably coupled to the portion (242) of the partition (240) which enables different assemblies to be used with the sample collection device (200). For example, an assembly (250) may be configured to allow a predetermined volume of sample to flow into the first chamber (216). The assembly (250) may be configured to permit fluid flow of a predetermined volume of sample (e.g., biofluid) from the second chamber (218) to the first chamber (216), and to resist fluid flow from the first chamber (216) to the second chamber (218). The port (254) may define one or more openings that may be configured to permit fluid flow from the second chamber (218) to the first chamber (216). As described in more detail herein, the flexible member (252) may be configured to transition between an open configuration and a closed configuration based on fluid flow through the port (254) and gas flow through the vent (257, 258). For example, the flexible member (252) may be configured to form a seal over the port (254) when the first chamber (216) holds a predetermined volume of sample (e.g., 20 ml to 30 ml).

The assembly (650) coupled to the partition (640) may comprise at least one port (654), a vent (657, 658), and a flexible member (652). The port (654) may be coupled to the partition (640). For example, the port (654) may couple to the central portion (644). The vent (657, 658) may be configured to allow one or more gases to escape from the first chamber. The flexible member (652) may be coupled to the port (654) and vent (657). For example, an assembly (650) may be configured to allow a predetermined volume of sample to flow into the first chamber (616). The assembly (650) may be configured to permit fluid flow of a predetermined volume of sample (e.g., biofluid) from the second chamber (618) to the first chamber (616), and to resist fluid flow from the first chamber (616) to the second chamber (618). The port (654) may define one or more openings that may be configured to permit fluid flow from the second chamber (618) to the first chamber (616). As described in more detail herein, the flexible member (652) may be configured to transition between an open configuration and a closed configuration based on fluid flow through the port (654) and gas flow through the vent (657, 658). For example, the flexible member (652) may be configured to form a seal over the port (654) when the first chamber (616) holds a predetermined volume of sample (e.g., 20 ml to 30 ml).

In some embodiments, a portion of the partition (640) facing the first chamber (616), as shown in FIGS. 6A, 6B, and 6E, may include a flat portion (648) configured to releasably contact the flexible member (not shown in FIG. 6E for the sake of clarity). For example, the flexible member (652) may be configured to form a seal in contact with the flat portion (648) when the first chamber (616) holds a predetermined volume of sample. The seal formed by the contact between the flexible member (652) and the flat portion (648) may cover and close at least one port (654).

Alternatively, in some embodiments, the sample collection device (600) may operate absent a flexible member (652). For example, a second lid (630) may be configured to seal one or more of the vent (658) and the at least one port (654) from the second chamber (618) when the second lid (630) is coupled to the enclosure (610).

FIGS. 3A-3C illustrate a fluid flow process through an assembly (320) of a sample collection device (300). The assembly may comprise a port (322) coupled to a flexible member (324), and a vent (326). The port (322) may define a set of openings (323). The assembly (320) may be disposed in an enclosure (310) so as to separate the device (300) into a first chamber (312) and a second chamber (314).

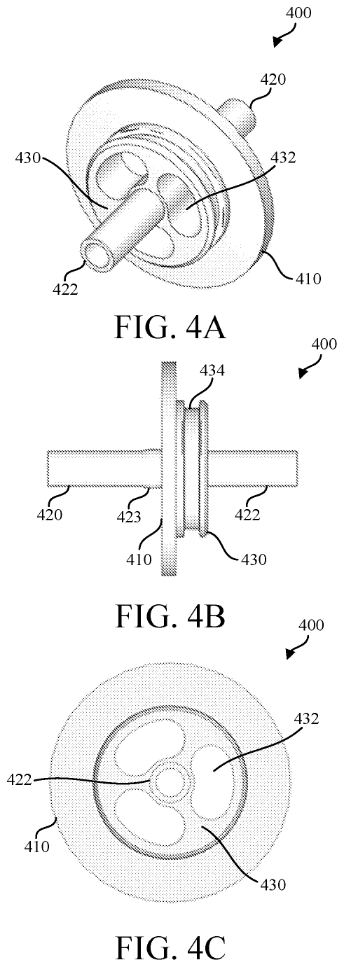

FIGS. 4A-4C illustrate another embodiment of an assembly (400) comprising a flexible member (410) coupled to a vent (420, 422), and a port (430). The vent (420, 422) may include a first chamber vent (420) and a second chamber vent (422). The first chamber vent (420) may define a protrusion (423). The port (430) may define a set of openings (432) and a recess (434) around a perimeter of the port (430).

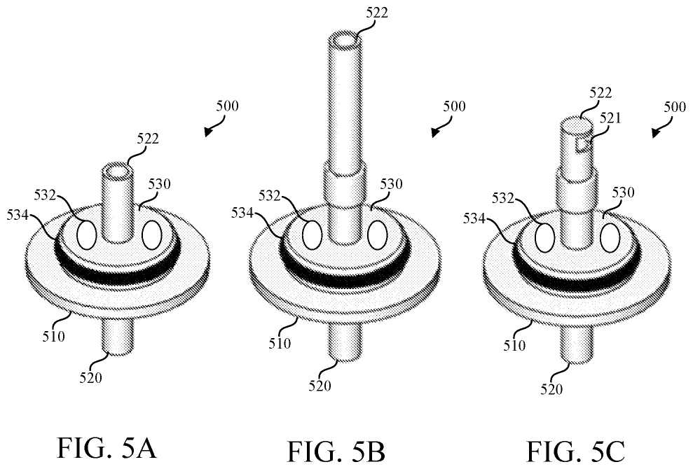

FIGS. 5A-5C illustrate yet another embodiment of an assembly (500) comprising a flexible member (510) coupled to a vent (520, 522), and a port (530), The vent (520, 522) may include a first chamber vent (520) and a second chamber vent (522). The port (530) may define a set of openings (532) and may be coupled to a sealing member (534) around a perimeter (e.g., circumference) of the port (530).

### Port

A sample collection device may include at least one port coupled to a partition and/or enclosure where the port defines an opening configured to allow unrestricted fluid flow from the second chamber to the first chamber. Control of fluid flow through one or more ports may be modulated by a flexible member and vent of the assembly. For example, FIGS. 3A-3C illustrate a set of three openings (323) of a port (322) where fluid (330) flows from a second chamber (314) into a first chamber (312). FIGS. 4A and 4C illustrate respective perspective and plan views of a port (430) of an assembly (400). The port (430) may define one or more openings (432) (e.g., through holes) configured for fluid flow. The shape, number, position, and size of the openings (432) may be configured to provide any suitable fluid flow rate. For example, FIG. 4C illustrates a set of three openings (432) having a generally uniform (e.g., kidney) shape and equidistant spacing. FIGS. 5A-5C illustrate variations of a port (530) having a pair of opposing ellipsoid openings (532).

In some embodiments, the port (430) may be removably engaged to the partition. As shown in FIG. 4B, the port (430) may define a recess (430) configured to engage a sealing member (not shown in FIG. 4B) such as a gasket and O-ring. For example, the recess (430) may be defined along a perimeter (e.g., sidewall) of the port (430). The assemblies (400) shown in FIGS. 4A-4C may be used in any of the sample collection devices (e.g., devices (100, 200, 300, 600)) described herein. FIGS. 5A-5C illustrate a sealing member (534) coupled to an outer perimeter of the port (530). A partition engaged with the port and a sealing member may be configured to form a fluid-tight seal such that fluid and gas flows between the chambers only through the openings and vent. A user may configure a device with an assembly having predetermined characteristics (e.g., fluid flow rate, sample volume).

### Flexible Member

A flexible member of an assembly may function as a valve to control fluid flow between the chambers of the enclosure. The flexible member may be configured with the flexibility to allow fluid flow through the port as well rigidity to prevent fluid flow between chambers when the first chamber holds a predetermined volume of fluid. For example, the flexible member may be flexible enough to bend to allow urine to flow into the first chamber during donation, but rigid enough to prevent fluid flow and leakage into the first chamber after donation of a predetermined volume. Furthermore, the flexible member is composed of a material inert to biological matter such that contact with the flexible member does not alter the sample in a manner that impacts the diagnostic analysis of the sample.

The flexible member (252, 652) in FIGS. 2A-2B and 6A-6B are shown in a closed configuration that may form a seal over respective ports (254, 654). The flexible member (252, 652) may transition between the closed configuration and an open configuration (FIG. 3B) where portions of the flexible member (252, 652) extend away from the port (254, 654) such that fluid may flow from the second chamber (218, 618) into the first chamber (216, 616). As shown in FIG. 3B, in the open configuration, an outer portion of the flexible member (324) bends further away from the port (323) than an inner portion of the flexible member (324). The flexible member (252, 652) is configured to be aligned over the port (254, 654) such that the flexible member (252, 654) may form a seal over the port (254, 654) in the closed configuration. In FIGS. 2A-2B and 6A-6B, the flexible member (252, 652) has a diameter greater than that of the port (254, 654).

The thickness of the flexible member (252, 652) may provide the combination of flexibility and rigidity sufficient to facilitate desired fluid flow characteristics through the assembly (250, 650). Generally, a higher thickness corresponds to reduced flexibility. In some embodiments, the flexible member (252, 652) has a thickness of between about 0.1 mm and about 1 mm, between about 0.1 mm and about 0.7 mm, between about 0.2 mm and about 0.4 mm, and between about 0.3 mm and about 0.6 mm, including all values and sub-ranges in-between.

The flexible member (252, 652) may have a diameter sufficient to at least cover each opening of a port (254, 654) in the closed configuration. As shown in FIGS. 2A-2B, 4A-4C, 5A-5C, and 6A-6B, a diameter of the flexible member may be greater than a diameter of its port. In some embodiments, the flexible member (252, 652) has a diameter of between about 20 mm and about 30 mm, between about 22 mm and about 25 mm, between about 23 mm and about 27 mm, including all values and sub-ranges in-between. In some embodiments, the flexible member (252, 652), as well as any component of the sample collection device (200, 600), may be formed of a material that is inert to biological material. For example, the flexible member (252, 652) in contact with urine may not interfere with a urine-derived exoRNA signal or cause RT-inhibition.

In some embodiments, the flexible member (252, 652) may be composed of silicone. For example, at least the surface of a component configured to contact the sample may be composed of the material that is inert to biological material. In some embodiments, one or more components of a sample collection device may be composed of one or more of high density polypropylene, polyethylene, medical grade plastic, and the like.

### Vent

A vent of an assembly may be configured to allow gas flow between the chambers and to control a volume of sample held in the first chamber. When the flexible member is in the open configuration (such as when fluid flows from the second chamber (218, 618) into the first chamber (216, 616)), one or more gases may escape from the first chamber (216, 616) to the second chamber (218, 618) via the vent (257, 258, 657, 658). The vent (257, 258, 657, 658) may define a lumen that extends substantially parallel to a longitudinal axis of the enclosure (210, 610). In some embodiments, the vent (257, 258, 657, 658) may have a diameter of at least about 5 mm. This diameter may reduce tension between the flexible member (252, 652) and the vent (257, 258, 657, 658) when coupling the flexible member (252, 652) to the vent (257, 258, 657, 658) such that the flexible member (252, 652) is able to maintain a seal over at least one port (254, 654) when the device (200, 600) is at rest, handled, transported, and/or manipulated. Higher tension between the flexible member (252, 652) and the vent (257, 258, 657, 658) due to a reduced vent diameter may allow leakage and mixing of the first volume and second volume of sample. The vent (257, 258, 657, 658) may extend into at least one of the first chamber (216, 616) and the second chamber (218, 618).

In some variations, a volume of fluid that may be held in the first chamber may be a function of a length of a first chamber vent. For example, a length of a first chamber vent (257, 657) may be configured to control the volume of sample held in the first chamber (216, 616). In some embodiments, the first chamber vent (257, 657) may be configured to extend into the first chamber (216, 616) by up to about 15 mm. A length the first chamber vent (257, 657) may be linearly correlated with the first sample volume of the first chamber (216, 616) such that a longer vent length may reduce the first sample volume. When a volume of fluid in the first chamber (216, 616) reaches and covers the first chamber vent (257, 657), gas may be prevented from escaping through the first chamber vent (257, 657) and the flexible member (252, 652) forms a seal such that additional fluid does not flow into the first chamber (216, 616). For example, a first chamber vent (257, 657) length of between about 7 mm and about 10 mm may allow a first volume of about 25 ml of sample to be held in the first chamber (216, 616). A first chamber vent (257, 657) length of about 16 mm may allow a first volume of about 12 ml of sample. In some embodiments, a length of a second chamber vent (257, 657) may be configured based on a second volume of sample.

In some embodiments, the shape of the vent (257, 258, 657, 658) may be configured to aid coupling of the flexible member (252, 652) to the vent (257, 258, 657, 658). For example, the first chamber vent (257, 657) may include a protrusion (259, 659) where the flexible member (252, 652) is coupled to the first chamber vent (258, 658) between the partition (240, 640) and the protrusion (259, 659). The protrusion (259, 659) may provide structural support to an inner portion of the flexible member (252, 652). Similarly, as shown in the side view of FIG. 4B, a flexible member (410) may be coupled between a protrusion (423) of the first chamber vent (420) and the port (430). The vent (420, 422) may extend generally perpendicularly to the port (430). In some embodiments, the vent (420), 422) may extend through a central portion of the assembly (400).

FIGS. 5A-5C illustrate variations of the assembly (500) having different vent configurations. The assemblies (500) shown in FIGS. 5A-5C may be used in any of the sample collection devices (e.g., devices (100, 200, 300, 600)) described herein. FIG. 5A illustrates a second chamber vent (522) having a first length. The second chamber vent (522) in FIG. 5B has a second length greater than the first length. In FIGS. 5A and 5B, the ends of the vent (520, 522) open along a longitudinal axis of the vent (520, 522). In FIG. 5C, the second chamber vent (522) has a closed end and an aperture defined in a sidewall of the vent (522). This configuration may reduce the volume of sample that may flow from the second chamber to the first chamber via the second chamber vent (520) when the sample is being received in the second chamber.

### Lid

FIGS. 2B and 6B illustrate respective sample collection devices (200, 600) including a first lid (220, 620) (e.g., bottom lid) removably coupled to a first end (212, 612) of the enclosure (210, 610) and a second lid (230, 630) (e.g., top lid) removably coupled to a second end (214, 614) of the enclosure (210, 610). FIG. 8 is a perspective view of a first lid (800).

In some embodiments, the first lid (220) may comprise a protrusion (232) aligned with an opening of the second chamber vent (257) and that extend into the first chamber (216). In some embodiments, the second lid (230) coupled to the enclosure (210) may be configured to form a second seal between the first chamber (216) and the second chamber (218). For example, the second lid (230) may comprise a protrusion (222) configured to seal one or more of the second chamber vent (257) and at least one port (254) of the assembly (250) when the second lid (230) is coupled to the enclosure (210). For example, the protrusion (232) may form a generally conical or cylindrical shape that extends to contact at least the port (227). The protrusion (222) may define a recess (224) configured to accommodate the vent (224). Thus, fluid separation between the first chamber (216) and the second chamber (218) may be enhanced by sealing openings in the port (254) and second chamber vent (258) with the protrusion (222).

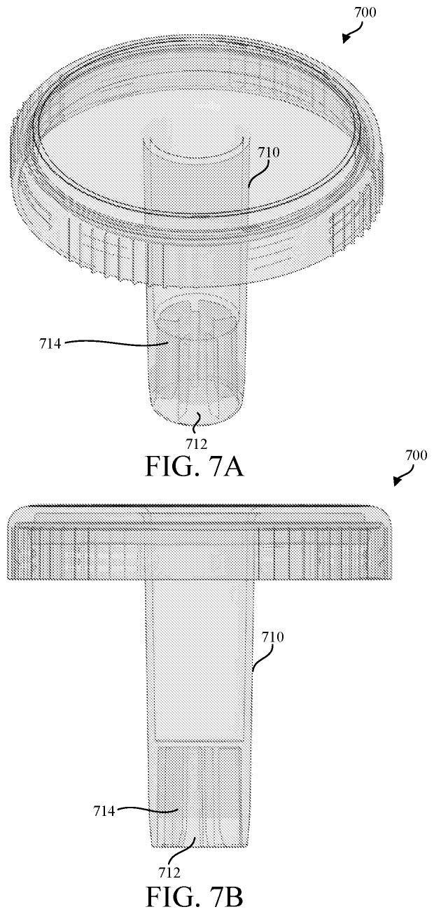

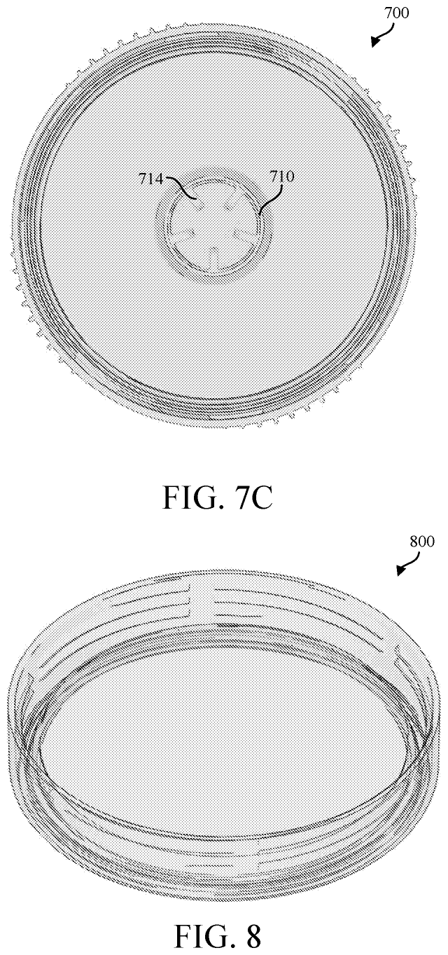

FIGS. 7A-7C are respective perspective, side, and plan views of a second lid (700). The second lid (700) may comprise a protrusion (710) configured to extend toward a partition of a sample collection device when the second lid (700) is coupled to an end of an enclosure. An end of the protrusion (710) may define a recess (712) configured to accommodate a vent. In some embodiments, a set of supports (714) may protrude from an inner circumference of the recess (712). The set of supports (714) may be configured to contact and hold a vent when the second lid is coupled to the sample collection device.

### II. Methods

Described herein are embodiments corresponding to methods for collecting a sample including a biofluid such as urine. These methods may allow different portions of a sample to be separated and collected and may in some embodiments be used with the systems and devices described herein. FIGS. 3A-3C are side cross-sectional views of a sample collection device (300) receiving a sample (350). FIG. 3A shows the sample collection device (300) prior to receiving a sample. In FIG. 3B, sample (330) flows into an open end of the second chamber (314) towards the assembly (320). As fluid (330) flows through the set of openings (323) from the second chamber (314) into the first chamber (312), gas (340) in the first chamber (312) is displaced and escapes from the first chamber (312) via the vent (326). As shown in FIG. 3B, the flow of fluid (340) places the flexible member (324) in an open configuration that bends the flexible member (324) away from one or more openings (323) of the port (322) such that a seal is not formed.

As shown in FIG. 3C, the first chamber (312) may hold a first volume (350) of sample. The first chamber (312) receives the sample until the sample covers (e.g., submerges) a first chamber end of the vent (326), at which point gas does not escape through the vent (326). At this point, the first chamber holds a first volume of sample and the flexible member (324) transitions to a closed configuration. The device (300) may continue to receive the sample where the second chamber (314) may hold a second volume (352) of the sample. It should be appreciated that the sample may be received continuously such that the device may automatically divide the sample into a first and second volume without user effort.

The flexible member (324) may be configured to resist fluid flow between the first chamber (312) and the second chamber (314) such that the first volume (350) and the second volume (352) remain separated in their respective chambers. For example, if the device (300) is tilted, rotated, handled, or otherwise manipulated, the flexible member (324) will remain in the closed configuration. This may allow, for example, a user to pour out a portion of the second volume (352) from the second chamber (314) while the flexible member (324) maintains the closed configuration such that the first and second volumes of the sample remain separated and do not mix.

In some embodiments, the first chamber may be configured to reduce and/or minimize the amount of first volume of sample that enters into the vent (326). For example, a partition of the device (300) may be concave so as to provide a recess where the first volume may collect when the device (300) is flipped upside down (e.g., where the flexible member (324) is above the port (322).

### Sample Collection

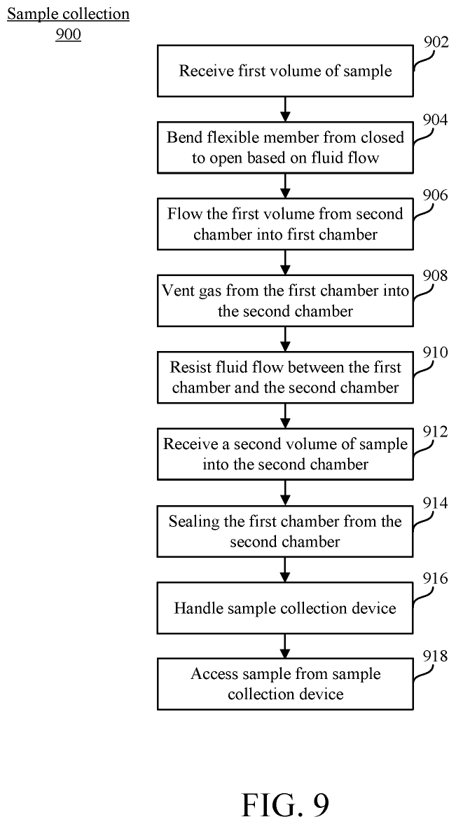

Methods for collecting a sample in some embodiments may use a sample collection device as described herein. FIG. 9 is a flowchart that generally describes a method of separating a sample (900). The process (900) may include step 902 of receiving a first volume (e.g., first catch) of the sample into a second chamber of an enclosure of a sample collection device (e.g., device (100, 200, 300, 600)). For example, the first volume of sample may contact a partition (640) of an enclosure (610) such that a predetermined amount of the first volume fills the recesses (646). In step 904, the flexible member bends so as to transition from the closed configuration to the open configuration based on the fluid flow. The flexible member in the open configuration bends away from the port. The flexible member in the open configuration permits the first volume to flow from the second chamber into the first chamber.

In step 906, the first volume may flow from the second chamber into a first chamber such that the flexible member bends, as shown for example in FIG. 3B. For example, the flexible member transitions to the open configuration such that the first volume flows into the second chamber and transitions to the closed configuration when the second volume flows into the second chamber.

In step 908, one or more gases from the first chamber may escape from the first chamber as the first volume flows into the first chamber. For example, a vent as described herein may allow gases to escape from the first chamber to the second chamber when displaced by the sample. The flexible member may be in the open configuration until an opening of the vent is blocked by the sample.

In step 910, the flexible member transitions from the open configuration to the closed configuration configured to resist fluid flow between the first chamber and the second chamber once a predetermined volume of sample is held in the first chamber. The flexible member is configured to form a seal over one or more ports when the first chamber holds a predetermined volume of sample and the vent is sealed by the sample. For example, the flexible member may be configured to form a seal in contact with a flat portion of the partition when the first chamber holds a predetermined volume of sample. In the closed configuration, the first volume in the first chamber is substantially separated from the second volume in the second chamber.

In step 912, a second volume of the sample may be received in the second chamber. For example, a user may continuously urinate into the second chamber without regard to the sample separation provided by the sample collection device. A central recess portion of the partition may initially fill with the second volume of the sample.

In step 914, the first chamber may be sealed from the second chamber by coupling a lid to the enclosure. For example, a second lid (230) may be screwed onto a second chamber end of the enclosure (210) to further seal the second chamber (218) from the first chamber (216). A first lid (220) may be coupled to the first end of the enclosure (210) prior to receiving the first volume of sample in step 902. In some embodiments, at least a portion of one of the lids may be transparent so as to permit visual inspection of the sample for characteristics such as color and clarity.

In step 916, the sample collection device may be handled while maintaining separation and integrity of the first and second volumes. In step 918, at least a portion of the sample from one of the first chamber and the second chamber may be accessed while maintaining separation and integrity of the first and second volumes. For example, at least a portion of the first volume from the first chamber may be accessed by rotating the sample collection device upside-down and removing a first lid. In some embodiments, the sample collection device may be tilted so that at least a portion (e.g., at least about 5 ml) of the first and second volumes of the sample may be poured out of the device and into another device (e.g., 50 ml “Falcon” tube, cup) while the separation between volumes is maintained. For example, the flexible member may maintain the closed configuration even when the device is tilted. In some embodiments, urinalysis may be performed using a test strip indicator (e.g., dip-stick, test stick) that may be inserted into a chamber of the device. The sample may undergo any suitable analysis, such as microscopic examination, assay testing, microbial culture (e.g., for urinary tract infection), combinations thereof, and the like.

Although some of the embodiments herein are described relative to urine collection and analysis, any of the illustrative devices, systems, and methods described herein may be used with any fluid such as water for microbe analysis and the like.

The foregoing description, for purposes of explanation, used specific nomenclature to provide a thorough understanding of various inventions and embodiments disclosed herein. However, it will be apparent to one skilled in the art that specific details are not required in order to practice the disclosed inventions and embodiments. Thus, the foregoing descriptions of specific embodiments of the inventions and corresponding embodiments thereof are presented for purposes of illustration and description. They are not intended to be exhaustive or to limit the invention to the precise forms disclosed; obviously, many modifications and embodiments are possible in view of the above teachings. The embodiments were chosen and described in order to best explain the principles of the inventions, the corresponding embodiments thereof, and practical applications, so as to enable others skilled in the art to best utilize the invention and various implementations with various modifications as are suited to the particular use contemplated. It is intended that the following claims and their equivalents define the scope of the invention.

In addition, any combination of two or more such features, structure, systems, articles, materials, kits, steps and/or methods, disclosed herein, if such features, structure, systems, articles, materials, kits, steps and/or methods are not mutually inconsistent, is included within the inventive scope of the present disclosure. Moreover, some embodiments of the various inventions disclosed herein may be distinguishable from the prior art for specifically lacking one or more features/elements/functionality found in a reference or combination of references (i.e., claims directed to such embodiments may include negative limitations).

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

What is claimed is:

1. A sample collection device, comprising:
an enclosure defining a first chamber, a second chamber, and a partition therebetween configured to separate the first chamber and the second chamber;
at least one port coupled to the partition;
a vent configured to allow one or more gases to escape from the first chamber; and
a flexible member coupled to the at least one port and configured to transition between an open configuration and a closed configuration based on fluid flow through the port and gas flow through the vent.

2. A sample collection device, comprising:
an enclosure defining a first chamber, a second chamber, and a partition therebetween configured to separate the first chamber and the second chamber; and
an assembly comprising at least one port, a sealing member, a vent, and a flexible member and configured to couple to a shoulder of the partition, the flexible member configured to transition between an open configuration and a closed configuration based on fluid and gas flow through the assembly.

3. A sample collection device, comprising:
an enclosure defining a first chamber, a second chamber, and a partition therebetween configured to separate the first chamber and the second chamber;
at least one port coupled to the partition;
a vent configured to allow one or more gases to escape from the first chamber; and
a lid comprising a protrusion configured to seal one or more of the vent and the at least one port from the second chamber when the lid is coupled to the enclosure.

4. The sample collection device of any of claims 1-3, wherein the open configuration is configured to permit fluid flow of a predetermined volume of sample from the second chamber to the first chamber, and the closed configuration is configured to resist fluid and gas flow from the first chamber to the second chamber.

5. The sample collection device of any of claims 1-4, wherein in the open configuration, one or more gases flow out of the first chamber toward the second chamber via the vent during the fluid flow from the second chamber into the first chamber.

6. The sample collection device of any of claims 1-5, wherein the flexible member in the open configuration bends away from the at least one port and the flexible member in the closed configuration forms a seal over the at least one port.

7. The sample collection device of any of claims 1-6, wherein the flexible member is configured to form a seal over the at least one port when the first chamber holds a predetermined volume of sample.

8. The sample collection device of any of claims 1-7, wherein the partition comprises a flat portion, and the flexible member is configured to form a seal in contact with the flat portion when the first chamber holds a predetermined volume of sample.

9. The sample collection device of any of claims 1-8, wherein the partition defines at least one recess around a circumference of the at least one port.

10. The sample collection device of any of claims 1-9, wherein the partition comprises a central recess portion.

11. The sample collection device of any of claims 1-10, wherein the flexible member has a thickness of between about 0.1 mm and about 1 mm.

12. The sample collection device of any of claims 1-11, wherein the flexible member has a diameter of between about 20 mm and about 30 mm.

13. The sample collection device of any of claims 1-12, wherein the flexible member is composed of silicone.

14. The sample collection device of any of claims 1-13, wherein the vent is configured to permit gas flow from the first chamber to the second chamber during fluid flow into the first chamber.

15. The sample collection device of any of claims 1-14, wherein the vent defines a lumen.

16. The sample collection device of any of claims 1-15, wherein the vent has a diameter of at least about 5 mm.

17. The sample collection device of any of claims 1-16, wherein the enclosure defines a longitudinal axis and the vent is substantially parallel to the longitudinal axis.

18. The sample collection device of any of claims 1-17, wherein the vent extends into at least one of the first chamber and the second chamber.

19. The sample collection device of any of claims 1-18, wherein the vent extends into the first chamber by up to about 15 mm.

20. The sample collection device of any of claims 1-19, wherein the vent extends into the second chamber, and a sidewall of the vent defines an aperture.

21. The sample collection device of any of claims 1-20, wherein the vent comprises a protrusion and the flexible member is coupled to the vent between the partition and the protrusion.

22. The sample collection device of any of claims 1-21, wherein the flexible member comprises a material inert to biological matter.

23. The sample collection device of any of claims 1-22, wherein the partition extends toward the first chamber.

24. The sample collection device of any of claims 1-23, wherein the partition is concave.

25. The sample collection device of any of claims 1-24, wherein the first chamber is configured to hold a sample volume of between about 5 ml and about 50 ml.

26. The sample collection device of any of claims 1-25, wherein the second chamber is configured to hold a sample volume of between about 10 ml and about 150 ml.

27. The sample collection device of any of claims 1-26, further comprising a first lid configured to removably couple to a first end of the enclosure.

28. The sample collection device of any of claims 1-27, further comprising a second lid configured to removably couple to a second end of the enclosure.

29. The sample collection device of claim 28, wherein the second lid coupled to the enclosure is configured to form a seal between the first chamber and the second chamber.

30. The sample collection device of claim 28, wherein the second lid comprises a protrusion configured to seal one or more of the vents and the at least one port from the second chamber when the second lid is coupled to the enclosure.

31. The sample collection device of claim 30, wherein the protrusion defines a recess configured to accommodate the vent.

32. The sample collection device of any of claims 1-31, wherein the port defines a recess configured to engage a sealing member.

33. A sample collection device, comprising:
an enclosure defining a first chamber, a second chamber, and a partition therebetween configured to separate the first chamber and the second chamber, wherein the enclosure defines a longitudinal axis, and the partition is concave and extends toward the first chamber;
at least one port coupled to the partition, and a sealing member therebetween;
a flexible member coupled to the at least one port;
a vent coupled to the flexible member, wherein the vent defines a lumen and extends substantially parallel to the longitudinal axis, wherein the vent comprises a first protrusion and the flexible member is coupled between the partition and the protrusion;
a first lid is coupled to a first end of the enclosure; and
a second lid comprises a second protrusion configured to seal one or more of the vent and the at least one port from the second chamber when the second lid is coupled to a second end of the enclosure, wherein the second protrusion defines a recess configured to accommodate the vent.

34. A sample collection method, comprising:
receiving a first volume of a sample into a sample collection device, the sample collection device comprising an enclosure defining a first chamber and a second chamber, the enclosure comprising a flexible member and a partition configured to separate the first chamber and the second chamber, wherein the first volume is received in the second chamber;
flowing the first volume from the second chamber to the first chamber such that the flexible member bends;
receiving a second volume of the sample in the second chamber; and
resisting fluid and gas flow between the first chamber and the second chamber such that the first volume in the first chamber is substantially separated from the second volume in the second chamber.

35. The method of claim 34, further comprising transitioning the flexible member between an open configuration and a closed configuration based on fluid and gas flow, wherein the open configuration permits fluid and gas flow from the second chamber to the first chamber, and the closed configuration resists fluid and gas flow from the first chamber to the second chamber.

36. The method of any of claim 34 or 35, further comprising handling the sample collection device while maintaining the separation between the first volume and the second volume.

37. The method of any of claims 34-36, further comprising sealing the first chamber from the second chamber by coupling a lid to the enclosure.

38. The method of any of claims 34-37, further comprising venting one or more gases from the first chamber to the second chamber.

39. The method of any of claims 34-38, further comprising accessing at least a portion of the sample from one of the first chamber and the second chamber while maintaining separation between the first volume and the second volume.

40. The method of claim 39, wherein accessing at least the portion of the first volume from the first chamber comprises tilting the sample collection device.

41. The method of any of claims 34-40, wherein the sample is urine.

42. A sample collection system or method according to any one or more embodiments disclosed herein.
